(19) United States
(12) Patent Application Publication
(10) Pub. No.: US 2022/0064681 A1
(43) Pub. Date: Mar. 3, 2022

(54) E. COLI VARIANT STRAIN OR CORYNEBACTERIUM GLUTAMICUM VARIANT STRAIN PRODUCING L-AMINO ACIDS, AND METHOD FOR PRODUCING L-AMINO ACIDS USING SAME

(71) Applicant: DAESANG CORPORATION, Seoul (KR)

(72) Inventors: Sun Hee LEE, Gyeonggi-do (KR); Hyun Ho KIM, Seoul (KR); Hyun Young KIM, Gyeonggi-do (KR); Young II JO, Seoul (KR); Yong Soo KIM, Gyeonggi-do (KR); Cheol Min YANG, Gyeonggi-do (KR); Won Joo SHIN, Incheon (KR)

(73) Assignee: DAESANG CORPORATION, Seoul (KR)

(21) Appl. No.: 17/418,072

(22) PCT Filed: Dec. 18, 2019
(86) PCT No.: PCT/KR2019/017934

§ 371 Date: Jun. 24, 2021

(30) Foreign Application Priority Data
Dec. 26, 2018 (KR) 10-2018-0169847

(51) Int. Cl. C12P 13/22; C12N 1/20; C12P 13/04

(52) U.S. Cl. CPC C12P 13/227; C12P 13/222; C12P 13/04; C12N 1/205

(57) ABSTRACT

The present disclosure relates to an L-amino acid-producing E. coli mutant strain or Corynebacterium glutamicum mutant strain having enhanced L-amino acid productivity, and a method of producing L-amino acid using the same. The L-amino acid-producing E. coli mutant strain and Corynebacterium glutamicum mutant strain according to the present disclosure exhibit an enhanced ability to produce L-amino acid, such as L-tryptophan, L-phenylalanine, L-tyrosine, L-glutamine, L-lysine, L-arginine, L-valine, L-leucine, L-isoleucine, L-threonine, L-histidine, L-serine, or L-citrulline, compared to parent strains thereof, and are capable of producing a high concentration of L-amino acid in high yield.

### TECHNICAL FIELD

The present disclosure relates to an E. coli mutant strain or Corynebacterium glutamicum mutant strain having enhanced L-amino acid productivity and a method of producing L-amino acid using the same.

### BACKGROUND ART

L-tryptophan is one of the essential amino acids, and is required to polymerize proteins or synthesize vitamins such as nicotinic acid amide in vivo, and is widely used as an amino acid fortifying agent, a feed additive, a raw material for pharmaceuticals such as an infusion solution, and a health food material. L-phenylalanine is one of the essential amino acids, and serves in vivo as a precursor of: the non-essential amino acid tyrosine; a neurotransmitter such as dopamine, norepinephrine or adrenaline; or the skin pigment melanin, and is widely used as a raw material for food, cosmetics, and pharmaceuticals. In addition, various amino acids such as L-tyrosine, L-glutamine, L-lysine, L-arginine, L-valine, L-leucine, L-isoleucine, L-threonine, L-histidine, L-serine, and L-citrulline are widely used in various fields such as food, cosmetics and pharmaceuticals.

These L-amino acids were industrially produced by fermentation methods using naturally occurring microbial strains or their mutant strains modified to have enhanced L-amino acid productivity. As the mutant strains, auxotrophic strains or regulatory region mutant strains of microorganisms such as Escherichia coli and Corynebacterium have been used. Since the 1980s, as genetic recombination technology has rapidly developed and metabolic processes and their regulatory mechanisms have been identified in detail, many researchers have achieved great results by developing excellent recombinant strains using genetic manipulation techniques.

When genetic recombination technology is used, it is possible to enhance amino acid productivity by increasing the activity of an enzyme involved in amino acid biosynthesis or by inhibiting feedback caused by the produced L-amino acid. In addition, it is possible to enhance amino acid productivity by regulating the expression of amino acid exporter genes. U.S. Pat. No. 5,972,663 describes artificially overexpressing genes such as mex, bmr and qacA in several strains (such as E. coli) to enhance the abilities of the strains to produce L-cysteine, L-cystine, N-acetylserine and thiazolidine derivatives. European Patent No. 1016710 describes artificially overexpressing the genes yahN, yeaS, yfiK and yggA in an Escherichia sp. strain to enhance the ability of the strain to produce L-glutamic acid, L-lysine, L-threonine, L-alanine, L-histidine, L-proline, L-arginine, L-valine and L-isoleucine. Korean Patent No. 10-1023925 describes artificially overexpressing the gene yddG in an Escherichia sp. strain to enhance the ability of the strain to produce L-tryptophan and L-phenylalanine.

Based on these prior studies, the present inventors have constructed L-amino acid-producing mutant strains, which have enhanced ability to produce various L-amino acids, by regulating the expression of L-amino acid exporter genes in an Escherichia sp. strain and a Corynebacterium sp. strain, thereby completing the present disclosure.

### Prior Art Documents

### [Patent Documents]

(Patent Document 1) U.S. Pat. No. 5,972,663

(Patent Document 2) European Patent No. 1016710

(Patent Document 3) Korean Patent No. 10-1023925

### DISCLOSURE

### Technical Problem

In order to the above-described problems,

an object of the present disclosure is to provide an Escherichia coli mutant strain having enhanced L-amino acid productivity due to overexpression of at least one gene selected from the group consisting of yicL, ydiN, ydhK, aaeB, yeeA, rhtC and emrD.

Another object of the present disclosure is to provide a Corynebacterium glutamicum mutant strain having enhanced L-amino acid productivity due to overexpression of at least one gene selected from the group consisting of yicL, ydiN, ydhK, aaeB, yeeA, rhtC and emrD.

Still another object of the present disclosure is to provide a method for producing L-amino acid, the method comprising steps of: culturing the mutant strain in a medium; and recovering L-amino acid from the mutant strain or the medium.

### Technical Solution

An L-amino acid-producing mutant strain according to one embodiment of the present disclosure may be an Escherichia coli mutant strain or Corynebacterium glutamicum mutant strain having enhanced L-amino acid productivity due to overexpression of at least one gene selected from the group consisting of yicL, ydiN, ydhK, aaeB, yeeA, rhtC and emrD.

In the prior art, there was a case in which the production of the aromatic amino acids L-tryptophan and L-phenylalanine was improved by regulating the amino acid exporter gene yddG in an Escherichia sp. mutant strain so as to be overexpressed. However, the present inventors examined the effect of the gene yddG on the production of L-amino acids, and as a result, confirmed that L-tryptophan and L-phenylalanine were still expressed in the strain from which the gene yddG was deleted (see Experimental Example 1). Thereby, the present inventors recognized that genes other than the gene yddG are involved in the production of amino acids. In addition, the present inventors selected a new amino acid exporter gene and constructed L-amino acid-producing mutant strains having enhanced ability to produce L-amino acids such as L-tryptophan, L-phenylalanine, L-tyrosine, L-glutamine, L-lysine, L-arginine, L-valine, L-leucine, L-isoleucine, L-threonine, L-histidine, L-serine and L-citrulline.

According to one embodiment of the present disclosure, the amino acid exporter gene may be one of genes represented by the nucleotide sequences of SEQ ID NOs: 1 to 7. Specifically, the amino acid exporter gene may be the gene yicL represented by the nucleotide sequence of SEQ ID NO: 1, the gene ydiN represented by the nucleotide sequence of SEQ ID NO: 2, the gene ydhK represented by the nucleotide sequence of SEQ ID NO: 3 or 37, the gene aaeB represented by the nucleotide sequence of SEQ ID NO: 4, the gene yeeA represented by the nucleotide sequence of SEQ ID NO: 5, the gene rhtC represented by the nucleotide sequence of SEQ ID NO: 6, or the gene emrD represented by the nucleotide sequence of SEQ ID NO: 7 or 39.

In addition, the amino acid exporter gene may be one of genes encoding the proteins represented by the amino acid sequences of SEQ ID NOs: 8 to 14. Specifically, the amino acid exporter gene may be a yicL protein represented by the amino acid sequence of SEQ ID NO: 8, a ydiN protein represented by the amino acid sequence of SEQ ID NO: 9, a ydhK protein represented by the amino acid sequence of SEQ ID NO: 10 or 38, an aaeB protein represented by the amino acid sequence of SEQ ID NO: 11, a yeeA protein represented by the amino acid sequence of SEQ ID NO: 12, an rhtC protein represented by the amino acid sequence of SEQ ID NO: 13, or an emrD protein represented by the amino acid sequence of SEQ ID NO: 14.

According to one embodiment of the present disclosure, the mutant strain may be selected from an herichia strain or a Corynebacterium sp. strain as a parent strain.

The Escherichia sp. strain is widely used for the production of several L-amino acids such as L-glutamic acid, L-lysine, L-threonine and L-cysteine, and is particularly known to have high L-tryptophan productivity. The parent strain is preferably Escherichia coli, which is easily available and has good convenience.

The Corynebacterium sp. strain has the advantage of low production of by-products while having high ability to produce L-amino acids such as L-glutamic acid and L-lysine, and is capable of stably producing amino acids even in a limited environment such as oxygen deficiency or sugar depletion. The parent strain is preferably Corynebacterium glutamicum which is mainly used to produce amino acids.

This parent strain may be a wild-type strain obtained in nature conditions or a strain in which the gene of the wild-type strain has been artificially manipulated.

In the present specification, the term “L-amino acid-producing Escherichia coli mutant strain” and “L-amino acid-producing Corynebacterium glutamicum mutant strain” refer to microorganisms mutated to have the ability to produce L-amino acids at a higher concentration than the parent strains. At this time, mutagenesis of the microorganisms may be performed by various means well known in the art, and one of physical or chemical mutagenesis methods may be used. For example, as chemical mutagens, N-methyl-N′-nitro-N-nitrosoguanidine (NTG), diepoxybutane, ethylmethane sulfonate, a mustard compound, hydrazine and nitrous acid may be used, but the chemical mutagens are not limited to these compounds. In addition, as physical mutagens, ultraviolet and gamma radiations may be used, but the physical mutagens are not limited thereto. Upon mutagenesis, the parent strain is affected by mutagens at a concentration sufficient to leave a surviving population having a certain size. The size may vary depending on the types of mutagenic factors, and depends on the amount of mutation that is induced in a surviving population at a constant killing rate by the mutagens. For example, when NTG is used, the killing rate may be 10% to 50% of the starting population. Mutagenesis by nitrous acid may be 0.01% to 0.1% of the starting population.

According to one embodiment of the present disclosure, the L-amino acids may be selected from the group consisting of L-tryptophan, L-phenylalanine, L-tyrosine, L-glutamine, L-lysine, L-arginine, L-valine, L-leucine, L-isoleucine, L-threonine, L-histidine, L-serine and L-citrulline, but are not limited thereto.

According to one embodiment of the present disclosure, the L-amino acid-producing Escherichia coli mutant strain and the L-amino acid-producing Corynebacterium glutamicum mutant strain may overexpress the amino acid exporter gene due to either transformation with the amino acid exporter gene or amplification of the number of copies of the amino acid exporter gene under the control of a strong promoter on the chromosome of each of the strains. According to one embodiment of the present disclosure, it is possible to obtain a mutant strain having enhanced L-amino acid productivity due to overexpression of the amino acid exporter gene compared to the parent strain, by inserting at least one of the seven amino acid exporter genes into a bacterial plasmid containing a promoter and introducing the plasmid into a parent strain.

Using this mutant strain, it is possible to produce a high concentration of L-amino acid in high yield. Specifically, a method for producing L-amino acid may comprise steps of: culturing the mutant strain in a medium; and recovering L-amino acid from the mutant strain or the medium.

In the present specification, the term “medium” refers to a medium which is used in culture of the Escherichia coli mutant strain and Corynebacterium glutamicum mutant strain of the present disclosure and contains carbon sources, nitrogen sources and inorganic salts so that the mutant strains are capable of producing a high yield of L-amino acid, for example, L-tryptophan, L-phenylalanine, L-tyrosine, L-glutamine, L-lysine, L-arginine, L-valine, L-leucine, L-isoleucine, L-threonine, L-histidine, L-serine or L-citrulline.

Examples of the carbon sources that may be used in the medium include, but are not limited to, saccharides and carbohydrates such as glucose, sugar, citrate, fructose, lactose, maltose or molasses; oils and fats, such as soybean oil, sunflower oil, castor oil or coconut oil; fatty acids such as palmitic acid, stearic acid or linoleic acid; glycerol; alcohols such as ethanol; and organic acids such as acetic acid. These substances may be used individually or as a mixture. Preferably, the medium for the Escherichia coli mutant strain may contain glucose. In addition, preferably, the medium for the Corynebacterium glutamicum mutant strain may contain glucose, molasses, or glucose and molasses. Most preferably, the medium may contain, as carbon sources, molasses in an amount of 5 to 60 parts by weight based on 100 parts by weight of glucose.

Examples of the nitrogen sources that may be used in the medium include, but are not limited to, peptone, meat extract, yeast extract, dried yeast, corn steep liquor, soybean cake, urea, thiourea, ammonium salt, nitrate, and other compounds organic or inorganic nitrogen. In addition, examples of inorganic salts that may be used in the medium include, but are not limited to, compounds containing magnesium, manganese, potassium, calcium, iron, zinc, cobalt, etc.

Meanwhile, examples of phosphorus sources that may be used in the medium include, but are not limited to, potassium dihydrogen phosphate or dipotassium hydrogen phosphate or the corresponding sodium-containing salts. In addition, the culture medium may contain metal salts such as magnesium sulfate or iron sulfate, which are required for growth. In addition, the above-described compounds may be used individually or as a mixture, but are not limited thereto.

Furthermore, in addition to the carbon source, nitrogen source and inorganic salt components, amino acids, vitamins, nucleic acids and compounds related thereto may be additionally added to the medium of the present disclosure. The above-described components may be added to the medium batchwise or in a continuous manner by a suitable method during culturing.

Meanwhile, the pH of the medium may be adjusted by adding a basic compound such as sodium hydroxide, potassium hydroxide or ammonia, or an acidic compound such as phosphoric acid or sulfuric acid in an appropriate manner. In addition, foaming may be suppressed using an anti-foaming agent such as a fatty acid polyglycol ester. To keep the medium in an aerobic condition, oxygen or an oxygen-containing gas (for example, air) may be injected into the medium.

In the present disclosure, the term “culturing” means growing a microorganism under artificially controlled suitable environmental conditions. Culturing of the microorganism of the present disclosure may be performed using the Escherichia coli or Corynebacterium glutamicum culturing method widely known in the art. Specifically, examples of the culturing method include, but are not limited to, batch culture, continuous culture, and fed-batch culture.

The temperature of the culturing may generally be, but not limited to, 20° C. to 45° C. The culturing may be continued until the largest possible amount of an L-amino acid such as L-tryptophan, L-phenylalanine, L-tyrosine, L-glutamine, L-lysine, L-arginine, L-valine, L-leucine, L-isoleucine, L-threonine, L-histidine, L-serine or L-citrulline is produced. The culturing may be generally performed for, but not limited to, 10 to 160 hours. The produced L-amino acid may be released into the culture medium or contained in the mutant strain.

The method for recovering L-amino acid from the mutant strain or the medium in which the mutant strain has been cultured may be performed using various methods widely known in the art, for example, but not limited to, centrifugation, filtration, anion exchange chromatography, crystallization and HPLC.

### Advantageous Effects

The L-amino acid-producing Escherichia coli mutant strain or Corynebacterium glutamicum mutant strain according to the present disclosure may exhibit enhanced L-amino acid productivity compared to the parent strain, and may produce a high concentration of L-amino acid in high yield.

### Mode for Invention

Hereinafter, the L-amino acid-producing Escherichia coli mutant strain or Corynebacterium glutamicum mutant strain according to the present disclosure and the method of producing L-amino acid using the same will be described in more detail with reference to examples. However, this description is provided by way of example only to aid the understanding of the present disclosure, and the scope of the present disclosure is not limited by this illustrative description.

### EXPERIMENTAL EXAMPLE 1-1

### Construction of yddG Gene-Deleted Mutant Strains

In order to examine the effect of the yddG gene on L-amino acid production, yddG gene-deleted mutant strains were constructed.

The yddG gene-deleted mutant strains were constructed using an L-tryptophan-producing E. coli W0G strain (accession number: KFCC11660P) and an L-phenylalanine-producing E. coli MWTR42 strain (accession number: KFCC10780) as parent strains with reference to the experimental method described in the literature (One-step inactivation of chromosomal genes in Escherichia coli K-12 using PCR products, Datsenko K A, Wanner B L., Proc Natl Acad Sci USA, 2000 Jun. 6; 97(12):6640-5).

First, to construct DNA fragments for disruption of the yddG gene, polymerase chain reaction (PCR) was performed using the chromosome of each parent strain and a pKD13 plasmid as a template and the primers shown in Table 1 below. At this time, the PCR was performed for a total of 30 cycles, each consisting of (i) denaturation at 95° C. for 30 sec, (ii) annealing at 58° C. for 30 sec, and (iii) extension at 72° C. for 2 min. The PCR products were electrophoresed on 0.8% agarose gel, and bands of desired size were obtained. Using the fragments corresponding to the bands, overlap PCR was performed under the above-described PCR conditions, thereby constructing single DNA fragments for disruption of the yddG gene. The DNA fragments were transformed into the respective parent strains which were then streaked on kanamycin-containing solid media and cultured at 37° C. for 24 hours. The resultant colonies were subjected to PCR under the above-described PCR conditions.

TABLE 1

| | Nucleotide sequence |
|---|---|
| Primer | (5′ → 3′) |
| yddG H1F | CAATGCCGCTACTGTTGTTCCAGCC |
| (SEQ ID | |
| NO: 15) | |
| yddG H1R | CAGTGGTGCGTTTTTCTACCGCTAT |
| (SEQ ID | |
| NO: 16) | |
| yddG H2F | AATAACTGCCGGGTCTACGGCC |
| (SEQ ID | |
| NO: 17) | |
| yddG H2R | AACGTATTTTCTAAACGAATTTTAAACGGCGTC |
| (SEQ ID | |
| NO: 18) | |
| yddG CF | CGGAACAGTATGTGCAGGTGTTACGG |
| (SEQ ID | |
| NO: 19) | |
| yddG CR | AACAAACCAGTTACAACCACCGCAAC |
| (SEQ ID | |
| NO: 20) | |

As a result, it was confirmed that the yddG gene was deleted in each colony.

In addition, the mutant strain obtained by deleting the yddG gene from the E. coli W0G strain was named W1G strain, and the mutant strain obtained by deleting the yddG gene from the E. coli MWTR42 strain was named MWTR5 strain.

### EXPERIMENTAL EXAMPLE 1-2

### Examination of L-Tryptophan and L-Phenylalanine Productivities of yddG Gene-Deleted Mutant Strains

The L-tryptophan productivities of the W0G and W1G strains and the L-phenylalanine productivities of the MWTR42 and MWTR5 strains were examined.

Each of the strains was 1% inoculated into a flask containing 10 ml of each medium having the composition shown in Table 2 below, and was cultured with shaking at 200 rpm at 37° C. for 70 hours. Each culture was measured for absorbance at OD₆₁₀ₙₘ, and the amount of L-tryptophan or L-phenylalanine produced was compared between the strains. The results are shown in Table 3 below.

TABLE 2

| Medium for tryptophan | | | Medium for phenylalanine | | |
|---|---|---|---|---|---|
| production | | | production | | |
| Component | Content | | Component | Content | |
| Glucose | 80.0 | g/L | Glucose | 80.0 | g/L |
| (NH₄)₂SO₄ | 20.0 | g/L | (NH₄)₂SO₄ | 20.0 | g/L |
| K₂HPO₄ | 0.8 | g/L | K₂HPO₄ | 1.0 | g/L |
| K₂SO₄ | 0.4 | g/L | KH₂PO₄ | 1.0 | g/L |
| MgCl₂ | 0.8 | g/L | K₂SO₄ | 0.4 | g/L |
| Fumaric acid | 1.0 | g/L | MgCl₂ | 1.0 | g/L |
| Yeast extract | 1.0 | g/L | Fumaric acid | 0.5 | g/L |
| (NH₄) ₆Mo₇O₂₄ | 0.12 | ppm | Yeast extract | 1.0 | g/L |
| H₃BO₃ | 0.01 | ppm | Glutamic acid | 0.5 | g/L |
| CuSO₄ | 0.01 | ppm | CaCl₂ | 5.00 | ppm |
| MnCl₂ | 2.00 | ppm | MnCl₂ | 2.00 | ppm |
| ZnSO₄ | 0.01 | ppm | ZnSO₄ | 1.00 | ppm |
| CoCl₂ | 0.10 | ppm | CoCl₂ | 0.10 | ppm |
| FeCl₂ | 10.00 | ppm | FeCl₂ | 10.00 | ppm |
| Thiamine_HCl | 20.00 | ppm | Thiamine_HCl | 20.00 | ppm |
| L-tyrosine | 200.00 | ppm | L-tyrosine | 200.00 | ppm |

| | | | | |
|---|---|---|---|---|
| L-phenylalanine | 300.00 | ppm | CaCO₃ | 3% |

| | | | |
|---|---|---|---|
| CaCO₃ | 3% | — | — |

TABLE 3

| Strain | L-tryptophan (g/L) | Strain | L-phenylalanine (g/L) |
|---|---|---|---|
| W0G | 4.21 ± 0.113 | MWTR42 | 21.3 ± 0.115 |
| W1G | 2.53 ± 0.132 | MWTR5 | 11.7 ± 0.141 |

As shown in Table 3 above, it was confirmed that the yddG-gene mutant strains (W1G strain and MWTR5 strain) still produced L-tryptophan and L-phenylalanine, respectively. That is, it could be seen that, even if yddG known as an aromatic amino acid exporter gene was deleted, the L-amino acid productivity of each of the mutant strains was retained, indicating that genes involved in amino acid export are present in addition to yddG.

### EXPERIMENTAL EXAMPLE 2-1

### Construction of Mutant Strains Containing the Amplified Genes yicL, ydiN, ydhK, aaeB, yeeA, rhtC and emrD, Respectively

E. coli mutant strains into which yicL, ydiN, ydhK, aaeB, yeeA, rhtC and emrD genes have been inserted, respectively, were constructed.

As a parent strain, an L-tryptophan-producing E. coli W0G strain (accession number: KFCC11660P) was used.

First, each of yicL, ydiN, ydhK, aaeB, yeeA, rhtC and emrD genes was amplified by PCR using the W0G strain as a template and the primers shown in Table 4 below, and then the PCR products were treated with the restriction enzymes SacI and XbaI, thereby preparing the genes.

As a result of sequencing, it was confirmed that the yicL gene has the nucleotide sequence of SEQ ID NO: 1, the ydiN gene has the nucleotide sequence of SEQ ID NO: 2, the ydhK gene has the nucleotide sequence of SEQ ID NO: 3 or 37, the aaeB gene has the nucleotide sequence of SEQ ID NO: 4, the yeeA gene has the nucleotide sequence of SEQ ID NO: 5, the rhtC gene has the nucleotide sequence of SEQ ID NO: 6, and the emrD gene has the nucleotide sequence of SEQ ID NO: 7 or 39.

The vector pTrc99A was digested by treatment with the restriction enzymes SacI and XbaI, and each of the prepared genes was inserted into the digested vector, thereby constructing the expression vectors pTrc99A::yicL, pTrc99A::ydiN, pTrc99A::ydhK, pTrc99A::aaeB, pTrc99A::yeeA, pTrc99A::rhtC, and pTrc99A::emrD. Each of the expression vectors was transformed into the W0G strain, thereby constructing the mutant strains W0G/pTrc99A::yicL, W0G/pTrc99A::ydiN, W0G/pTrc99A::ydhK, W0G/pTrc99A::aaeB, W0G/pTrc99A::yeeA, W0G/pTrc99A::rhtC, and W0G/pTrc99A::emrD, which contain each of the amplified genes.

TABLE 4

| | Nucleotide sequence |
|---|---|
| Primer | (5′ → 3′) |
| yicL_F | GCGAGCTCATGGGTTCCACCAGAAAGGG |
| (SEQ ID | |
| NO: 21) | |
| yicL_R | GCTCTAGATCACTTATGCCGCGCCGGA |
| (SEQ ID | |
| NO: 22) | |
| ydiN_F | GCGAGCTCATGTCTCAAAATAAGGCTTTCAGCA |
| (SEQ ID | |
| NO: 23) | |
| ydiN_R | GCTCTAGAGGCCATCAACCCAATCAATT |
| (SEQ ID | |
| NO: 24) | |
| ydhK_F | GCGAGCTCATGAACGCATCGTCATGGTCCTTGC |
| (SEQ ID | |
| NO: 25) | |
| ydhK_R | GCTCTAGATCACTTATGCCGCGCCGGA |
| (SEQ ID | |
| NO: 26) | |
| aaeB_F | GCGAGCTCATGGGTATTTTCTCCATTGCT |
| (SEQ ID | |
| NO: 27) | |
| aaeB_R | GCTCTAGATTTTGACTTAACTATCGGTca |
| (SEQ ID | |
| NO: 28) | |
| yeeA_F | GCGAGCTCGTGCGTGCCGATAAGTC |
| (SEQ ID | |
| NO: 29) | |
| yeeA_R | GCTCTAGATTATTTGCGCAAGGCCCG |
| (SEQ ID | |
| NO: 30) | |
| rhtC_F | GCGAGCTCATGTTGATGTTATTTCTCACCGT |
| (SEQ ID | |
| NO: 31) | |
| rhtC_R | gctctagaCTGGCATCACCGCGAAATAA |
| (SEQ ID | |
| NO: 32) | |
| emrD_F | GCGAGCTCATGAAAAGGCAAAGAAACG |
| (SEQ ID | |
| NO: 33) | |
| emrD_R | GCTCTAGACGGTGACGTGCGCTTAAAC |
| (SEQ ID | |
| NO: 34) | |

### EXPERIMENTAL EXAMPLE 2-2

### Examination of L-Tryptophan Productivities of Mutant Strains Containing the Amplified Genes yicL, ydiN, ydhK, aaeB, yeeA, rhtC and emrD, Respectively

The L-tryptophan productivities of W0G/pTrc99A obtained by inserting only the vector pTrc99A into W0G, and the mutant strains W0G/pTrc99A::yicL, W0G/pTrc99A::ydiN, W0G/pTrc99A::ydhK, W0G/pTrc99A::aaeB, W0G/pTrc99A::yeeA, W0G/pTrc99A::rhtC, and W0G/pTrc99A::emrD, were examined.

Each of the strains was 1% inoculated into a flask containing 10 ml of each medium having the composition shown in Table 2 above, and was cultured with shaking at 200 rpm at 34° C. for 72 hours. Each culture was measured for absorbance at OD₆₁₀ₙₘ, and the amount of L-tryptophan produced was compared between the strains. The results are shown in Table 5 below.

TABLE 5

| Strain | L-tryptophan (g/L) |
|---|---|
| W0G/pTrc99A::(control) | 4.21 ± 0.113 |
| W0G/pTrc99A::yicL | 4.83 ± 0.102 |
| W0G/pTrc99A::ydiN | 4.86 ± 0.169 |
| W0G/pTrc99A::ydhK | 4.88 ± 0.133 |
| W0G/pTrc99A::aaeB | 4.91 ± 0.101 |
| W0G/pTrc99A::yeeA | 4.95 ± 0.123 |
| W0G/pTrc99A::rhtC | 5.02 ± 0.131 |
| W0G/pTrc99A::emrD | 5.26 ± 0.156 |

As shown in Table 5 above, it was confirmed that the L-tryptophan productivities of the E. coli mutant strains containing the amplified genes yicL, ydiN, ydhK, aaeB, yeeA, rhtC and emrD, respectively, increased compared to that of the control.

### EXPERIMENTAL EXAMPLE 3-1

### Construction of Mutant Strains Containing Amplified Genes yicL, ydiN, ydhK, aaeB, yeeA, rhtC and emrD, Respectively

E. coli mutant strains into which yicL, ydiN, ydhK, aaeB, yeeA, rhtC and emrD genes were inserted, respectively, were constructed.

Expression vectors were constructed in the same manner as in Experimental Example 2-1 above, except that an L-phenylalanine-producing E. coli MWTR42 strain (accession number: KFCC10780) was used instead of the W0G strain. Each of the expression vectors was transformed into the MWTR42 strain, thereby constructing the mutant strains MWTR42/pTrc99A::yicL, MWTR42/pTrc99A::ydiN, MWTR42/pTrc99A::ydhK, MWTR42/pTrc99A::aaeB, MWTR42/pTrc99A::yeeA, MWTR42/pTrc99A::rhtC, and MWTR42/pTrc99A::emrD, which contain each of the amplified genes.

### EXPERIMENTAL EXAMPLE 3-2

### Examination of L-Phenylalanine Productivities of Mutant Strains Containing the Amplified Genes yicL, ydiN, ydhK, aaeB, yeeA, rhtC and emrD, Respectively

The L-phenylalanine productivities of MWTR42/pTrc99A obtained by inserting only the vector pTrc99A into MWTR42, and the mutant strains MWTR42/pTrc99A::yicL, MWTR42/pTrc99A::ydiN, MWTR42/pTrc99A::ydhK, MWTR42/pTrc99A::aaeB, MWTR42/pTrc99A::yeeA, MWTR42/pTrc99A::rhtC, and MWTR42/pTrc99A::emrD, were examined.

The amount of L-phenylalanine produced was compared between the strains in the same manner as in Experimental Example 2-2, and the results are shown in Table 6 below.

TABLE 6

| Strain | L-phenylalanine (g/L) |
|---|---|
| MWTR42/pTrc99A (control) | 21.3 ± 0.115 |
| MWTR42/pTrc99A::yicL | 27.1 ± 0.081 |
| MWTR42/pTrc99A::ydiN | 27.2 ± 0.165 |
| MWTR42/pTrc99A::ydhK | 27.3 ± 0.105 |
| MWTR42/pTrc99A::aaeB | 27.0 ± 0.111 |
| MWTR42/pTrc99A::yeeA | 27.3 ± 0.103 |
| MWTR42/pTrc99A::rhtC | 26.7 ± 0.122 |
| MWTR42/pTrc99A::emrD | 27.1 ± 0.085 |

As shown in Table 6 below, it was confirmed that the L-phenylalanine productivities of the E. coli mutant strains containing the amplified genes yicL, ydiN, ydhK, aaeB, yeeA, rhtC and emrD, respectively, increased compared to that of the control.

### EXPERIMENTAL EXAMPLE 4-1

### Construction of Mutant Strains Containing the Amplified Genes yicL, ydiN, ydhK, aaeB, yeeA, rhtC and emrD, Respectively

Corynebacterium glutamicum mutant strains into which yicL, ydiN, ydhK, aaeB, yeeA, rhtC and emrD genes have been inserted, respectively, were constructed.

As a parent strain, L-tryptophan-producing Corynebacterium glutamicum DW28G (accession number: KTCT13769BP) was used.

First, each of yicL, ydiN, ydhK, aaeB, yeeA, rhtC and emrD genes was amplified by PCR using each of the expression vectors (pTrc99A::yicL, pTrc99A::ydiN, pTrc99A::ydhK, pTrc99A::aaeB, pTrc99A::yeeA, pTrc99A::rhtC, and pTrc99A::emrD) constructed in Experimental Examples 2-1 as a template and the primers shown in Table 7 below, and then the PCR products were 5′-phosphorylated, thereby preparing the genes. The vector pEKO was digested by treatment with the restriction enzyme Eco53KI, and each of the prepared genes was inserted into the digested vector, thereby constructing the expression vectors pEKO::yicL, pEKO::ydiN, pEKO::ydhK, pEKO::aaeB, pEKO::yeeA, pEKO::rhtC, and pEKO::emrD. Each of the expression vectors was transformed into the DW28G strain, thereby constructing the mutant strains DW28G/pEKO::yicL, DW28G/pEKO::ydiN, DW28G/pEKO::ydhK, DW28G/pEKO::aaeB, DW28G/pEKO::yeeA, DW28G/pEKO::rhtC, and DW28G/pEKO::emrD, which contain each of the amplified genes.

TABLE 7

| | Nucleotide sequence |
|---|---|
| Primer | (5′ → 3′) |
| Primer_F | gcgccgacatcataacggttctgg |
| (SEQ ID NO: 35) | |
| Primer_R | cgcaacgttcaaatccgctcccg |
| (SEQ ID NO: 36) | |

### EXPERIMENTAL EXAMPLE 4-2

### Examination of L-Tryptophan Productivities of Mutant Strains Containing the Amplified Genes yicL, ydiN, ydhK, aaeB, yeeA, rhtC and emrD, Respectively

The L-tryptophan productivities of DW28G/pEKO obtained by inserting only the vector pEKO into DW28G, and the mutant strains DW28G/pEKO::yicL, DW28G/pEKO::ydiN, DW28G/pEKO::ydhK, DW28G/pEKO::aaeB, DW28G/pEKO::yeeA, DW28G/pEKO::rhtC, and DW28G/pEKO::emrD, were examined.

The amount of L-tryptophan produced was compared between the strains in the same manner as in Experimental Example 2-2, except that the composition shown in Table 8 was used as a medium. The results are shown in Table 9 below.

TABLE 8

| Component | Content | |
|---|---|---|
| Cane molasses (glucose) | 100.00 | g/L |
| KH₂PO₄ | 5.00 | g/L |
| K₂HPO₄ | 5.00 | g/L |
| K₂SO₄ | 0.40 | g/L |
| MgSO₄ | 0.25 | g/L |
| (NH₄) ₂SO₄ | 20 | g/L |
| Corn steep liquor | 10 | g/L |

| | |
|---|---|
| CaCO₃ | 3% |

TABLE 9

| Strain | L-tryptophan (g/L) |
|---|---|
| DW28G/pEKO (control) | 2.64 ± 0.107 |
| DW28G/pEKO::yicL | 3.57 ± 0.115 |
| DW28G/pEKO::ydiN | 3.05 ± 0.089 |
| DW28G/pEKO::ydhK | 3.11 ± 0.095 |
| DW28G/pEKO::aaeB | 2.95 ± 0.102 |
| DW28G/pEKO::yeeA | 3.21 ± 0.113 |
| DW28G/pEKO::rhtC | 3.31 ± 0.091 |
| DW28G/pEKO::emrD | 3.45 ± 0.115 |

As shown in Table 9 above, it was confirmed that the L-tryptophan productivities of the Corynebacterium glutamicum mutant strains containing the amplified genes yicL, ydiN, ydhK, aaeB, yeeA, rhtC and emrD, respectively, increased compared to that of the control.

In conclusion, in the present disclosure, the E. coli mutant strain or Corynebacterium glutamicum mutant strain having enhanced L-tryptophan or L-phenylalanine productivity compared to the parent strain was obtained by inducing overexpression of the amino acid exporter gene yicL, ydhK, aaeB, yeeA, rhtC or emrD, and it was confirmed that a high concentration of L-tryptophan or L-phenylalanine could be produced in high yield by using this mutant strain.

So far, the present disclosure has been described with reference to the embodiments thereof. Those of ordinary skill in the art to which the present disclosure pertains will appreciate that the present disclosure may be embodied in modified forms without departing from the essential characteristics of the present disclosure. Therefore, the disclosed embodiments should be considered from an illustrative point of view, not from a restrictive point of view. The scope of the present disclosure is defined by the claims rather than the foregoing description, and all differences within the scope equivalent thereto should be construed as being included in the present disclosure.

1. A mutant strain having enhanced L-amino acid productivity due to overexpression of at least one gene selected from the group consisting of yicL, ydiN, ydhK, aaeB, yeeA, rhtC and emrD, the mutant strain being Escherichia coli or Corynebacterium glutamicum.

2. The mutant strain of claim 1, wherein the yicL consists of the nucleotide sequence of SEQ ID NO: 1, the ydiN consists of the nucleotide sequence of SEQ ID NO: 2, the ydhK consists of the nucleotide sequence of SEQ ID NO: 3 or 37, the aaeB consists of the nucleotide sequence of SEQ ID NO: 4, the yeeA consists of the nucleotide sequence of SEQ ID NO: 5, the rhtC consists of the nucleotide sequence of SEQ ID NO: 6, and the emrD consists of the nucleotide sequence of SEQ ID NO: 7 or 39.

3. The mutant strain of claim 1, wherein the L-amino acid is selected from the group consisting of L-tryptophan, L-phenylalanine, L-tyrosine, L-glutamine, L-lysine, L-arginine, L-valine, L-leucine, L-isoleucine, L-threonine, L-histidine, L-serine and L-citrulline.

4. A method for producing L-amino acid, the method comprising steps of:
(a) culturing the mutant strain of claim 1 in a medium; and
(b) recovering L-amino acid from the mutant strain or the medium.

5. The method of claim 4, wherein the L-amino acid is selected from the group consisting of L-tryptophan, L-phenylalanine, L-tyrosine, L-glutamine, L-lysine, L-arginine, L-valine, L-leucine, L-isoleucine, L-threonine, L-histidine, L-serine and L-citrulline.


SEQUENCE LISTING (39 sequences)

SEQ ID NO: 1 - DNA, 924 bp, Artificial Sequence
  amino acid exporter gene yicL
atgggttccaccagaaaggggatgctgaacgttctgattgccgccgtgttgtggggaagttcaggggtctgcgcgcaatacatcatggagcaaagccagatgtcgtcgcagtttttgactatgacgcgtttgatattcgccggtttgattctactgacgctgtcatttgttcatggcgataaaatcttttctattattaacaatcataaagatgccattagcctgctgattttttccgtggttggcgcgctaactgtacagctcacttttttgctaaccatcgaaaaatcgaacgcagccacggcaacggtgctgcaattcctctcaccgacgattatcgtcgcctggttctcactggtgcgtaaatcgcgcccgggcattctggttttctgcgctattttgacatcgctggtcgggacttttttattggtgacacacggtaatccgacgtcattatcgatctctcctgccgcgttgttctggggcattgcctcggcatttgctgctgcattctataccacctatccctcaacgctaattgcccgctatggcacgttaccagtcgtcggctggagtatgctgattggcggtctgattctgttgcctttttatgccagacaaggaacaaactttgtcgttaacggcagtttgattctggcgtttttttatttggtggtcattggtacgtccctgacatttagtctgtacctgaaaggagcacaattaattggcggtccaaaagccagcattttgagctgtgcagaaccattaagtagcgcgctactctctttgctgttgctggggatcacgttcacattaccggactggctgggaacgctgctgattctgtcatcggtgattttgatttcaatggattcccgtcgccgcgccagaaaaataaatcgtccggcgcggcataagtga

SEQ ID NO: 2 - DNA, 1266 bp, Artificial Sequence
  amino acid exporter gene ydiN
atgtctcaaaataaggctttcagcacgccatttatcctggctgttctttgtatttacttcagctacttcctgcacggcattagtgttattacgcttgcccaaaatatgtcatctctggcggaaaagttttccactgacaacgcgggcattgcctacttaatttccggtatcggtttggggcgattgatcagtattttattcttcggtgtgatctccgataagtttggtcgtcgggcggtgatattaatggcagtaataatgtatctgctattcttctttggtattcccgcttgcccgaatttaactctcgcctacggtctggcagtgtgcgtaggtatcgctaactcagcgctggatacgggtggctaccccgcgctcatggaatgctttccgaaagcctctggttcggcggtcatactggttaaagcgatggtgtcatttgggcaaatgttctacccaatgctggtgagctatatgttgctcaataatatctggtacggctatgggctgattattccgggtattctatttgtactgatcacgctgatgctgttgaaaagcaaattccccagccagttggtggacgccagcgtaactaatgaattaccgcaaatgaacagcaaaccgttagtctggctggaaggtgtttcatcggtactgttcggtgtagccgcattctcgaccttttatgtgattgtggtgtggatgcccaaatatgcgatggcttttgctggtatgtcagaagctgaggcattaaaaaccatctcttattacagtatgggctcgttggtctgtgtctttatttttgccgcactactgaaaaaaatggtccggcccatctgggctaatgtatttaactctgcactggcaacaataacagcagccattatctacctgtacccttctccactggtgtgcaatgccggagcctttgttatcggtttctcagcagctggcggcattttacagctcggcgtttcggtcatgtcagagttttttcccaaaagcaaagccaaagtcaccagtatttatatgatgatgggtggactggctaactttgttattccactgattaccggttatctgtcgaacatcggcctgcaatatatcattgttctcgattttactttcgcgctgctggccctgattaccgcaattattgtttttatccgctattaccgcgttttcattattcctgaaaatgatgtgcggtttggcgagcgtaaattttgcacccggttaaacacaattaagcatagaggttaa

SEQ ID NO: 3 - DNA, 2013 bp, Artificial Sequence
  amino acid exporter gene ydhK
atgaacgcatcgtcatggtccttgcgcaatttgccctggttcagggccacgctggcgcaatggcgttatgcgttacgcaataccattgccatgtgtctggcgctgacggttgcctattatttaaatctggatgaaccctattgggcgatgacctcggctgcagtggttagctttcccaccgttggcggtgttatcagcaaaagcctcggacgcatcgctggcagtttgctcggagccattgcggcactgcttcttgccgggcatacgctcaatgagccgtggttttttctattgagcatgtcggcgtggcttggcttttgtacctgggcctgtgcgcacttcacgaataacgtcgcgtatgcatttcaactggcgggctacacggctgccatcatcgcctttccgatggttaatattactgaggccagccagctgtgggatatcgctcaggcgcgcgtttgcgaggtaatagtcggtattttgtgcggcggcatgatgatgatgatcctgccgagcagttccgatgctactgcccttttaaccgcattgaaaaacatgcacgcccgattactggaacatgccagtttactctggcagcctgaaacaaccgatgccattcgtgcagcacatgaaggggtgattgggcagatactgaccatgaatttgctgcgtatccaggctttctggagccactatcgttttcgccagcaaaacgcgcgccttaatgcgctgctccaccagcaattacgtatgaccagtgtcatctccagcctgcgacgtatgttgctcaactggccctcaccgccaggtgccacacgagaaattctcgaacagttgctgacggcgctcgccagttcgcaaacagatgtttacaccgtcgcacgtattatcgccccgctacgcccgaccaacgtcgccgactatcggcacgtcgccttctggcagcgactacgttatttttgccgcctttatctgcaaagtagtcaggaattacatcgtctgcaaagcggtgtagatgatcataccagactcccacggacatccggcctggctcgtcataccgataacgccgaagctatgtggagcgggctgcgtacattttgtacgttgatgatgattggcgcatggagtattgcttcgcaatgggatgccggtgccaatgcattaacgctggcagcaattagctgcgtactctactccgccgtcgcagcaccgtttaagtcgttgtcacttctgatgcgcacgctggtgttactttcgctattcagctttgtggtcaaatttggtctgatggtccagattagcgatctgtggcaatttttactgtttctctttccactgctggcgacaatgcagcttcttaaattgcagatgccaaaatttgccgcattgtgggggcaactgattgtttttatgggttcttttatcgctgtcactaatcccccggtgtatgattttgctgattttcttaacgataatctggcaaaaatcgttggcgtcgcgttggcgtggttagcgttcgccattctgcgtccaggatcggatgctcgtaaaagccgccgccatattcgcgcgctgcgccgggattttgtcgatcagctaagccgccatccaacactgagtgaaagcgaatttgaatcgctcacttatcatcacgtcagtcagttgagtaacagccaggatgcgctggctcgccgttggttattacgctggggtgtagtgctgctgaactgttctcatgttgtctggcaattgcgcgactgggaatcgcgttccgatccgttatcgcgagtacgggataactgtatttcactgttgcggggagtgatgagtgagcgtggcgttcagcaaaaatcactggcggccacacttgaagaattacagcggatttgcgacagccttgcccgtcatcatcaacctgccgcccgtgagctggcggcaattatctggcggctgtactgctcgctttcgcaacttgagcaagcaccaccgcaaggtacgctggcctcttaa

SEQ ID NO: 4 - DNA, 1968 bp, Artificial Sequence
  amino acid exporter gene aaeB
atgggtattttctccattgctaaccaacatattcgctttgcggtaaaactggcgaccgccattgtactggcgctgtttgttggctttcacttccagctggaaacgccacgctgggcggtactgacagcggcgattgttgccgccggtacggcctttgctgcgggaggtgaaccgtattctggcgctattcgctatcgtggctttttgcgcatcatcggcacatttattggctgtattgccggactggtgatcatcattgcgatgatccgcgcaccattattgatgattctggtgtgctgtatctgggccggtttttgtacctggatatcctcgctggtacgaatagaaaactcgtatgcgtgggggctggccggttataccgcgctgatcattgtgatcaccattcagccggaaccattgcttacgccgcagtttgccgtcgaacgttgtagcgagatcgttatcggtattgtgtgtgcgattatggcggatttgctcttttctccgcgatcgatcaaacaagaagtggatcgagagctggaaagtttgctggtcgcgcaatatcaattaatgcaactctgtatcaagcatggcgatggtgaagttgtcgataaagcctggggcgacctggtgcgacgcaccacggcgctacaaggcatgcgcagcaacctgaatatggaatcttcccgctgggcgcgggccaatcgacgtttaaaagcgatcaatacgctatcgctgacgctgattacccaatcctgcgaaacttatcttattcagaatacgcgcccggaattgatcactgatactttccgcgaattttttgacacgccggtagaaaccgcgcaggacgtccacaagcagctcaaacgcctgcggagagttatcgcctggaccggggaacgggaaacgcctgtcaccatttatagctgggtcgcggcggcaacgcgttatcagcttctcaagcgcggcgttatcagtaacacaaaaatcaacgccaccgaagaagagatcctgcaaggcgaaccggaagtaaaagtagagtcagccgaacgtcatcatgcaatggttaacttctggcgaaccacactttcctgcattctgggcacgcttttctggctgtggacgggctggacttccggcagtggtgcaatggtgatgattgcggtagtgacgtcactggcaatgcgtttgccgaatccacgcatggtggcgatcgactttatctacgggacgctggccgcgctgccgttagggctgctctactttttggtgattatccctaatacccaacagagcatgttgctgctgtgcattagcctggcagtgctgggattcttcctcggtatagaagtacagaaacggcgactgggctcgatgggggcactggccagcaccataaatattatcgtgctggataacccgatgactttccatttcagtcagtttctcgacagcgcattagggcaaatcgtcggctgtgtgctcgcgttcaccgttattttgctggtgcgggataaatcgcgcgacaggaccggacgtgtactgcttaatcagtttgtttctgccgctgtttccgcgatgactaccaatgtggcacgtcgtaaagagaaccacctcccggcactttatcagcagctgtttttgctgatgaataagttcccaggggatttgccgaaatttcgcctggcgctgacgatgattatcgcgcaccagcgcctgcgtgatgcaccgatcccggttaacgaggatttatcggcgtttcaccgacaaatgcgccgcacagcagaccatgtgatatctgcccgtagcgatgataaacgtcgtcggtactttggccagttgctggaagaactggaaatctaccaggaaaagctacgcatctggcaagcgccaccgcaggtgacggaaccggtaaatcggctggcggggatgctccataagtatcaacatgcgttgaccgatagttaa

SEQ ID NO: 5 - DNA, 1059 bp, Artificial Sequence
  amino acid exporter gene yeeA
gtgcgtgccgataagtcattaagcccgtttgaaatccgggtataccgccattaccgcattgtgcatggtactcgggtcgcgctggcattcctgctcacttttctcattatccgcctgtttactatcccggaaagcacctggccgctggtcaccatggtggtgattatggggccaatctcgttctggggtaacgttgtccctcgcgcctttgagcgtattggcggtacggtgttgggttcgattttaggtcttatcgctctgcaactggagttaatctcgttaccgctgatgttagtctggtgcgcggcggccatgttcctttgcggttggctggcgctgggcaagaaaccgtatcaaggtttattgattggggtgacgctggcaattgttgtgggttccccgacaggtgaaattgatacggcgttatggcgaagcggcgatgtgatcctcggctctttactggcaatgttgtttaccggtatctggccacaacgggcgttcatccactggcgcattcaactggcgaaaagtctgaccgagtataatcgggtctatcaatctgcattctcaccgaacttactcgaacgcccacgtctggaaagccatctacaaaaactcctgaccgatgccgtgaaaatgcgtggactgattgcgcccgccagcaaagaaacccgtattccaaaatcgatatatgaaggtatccagaccattaaccgcaatctggtttgtatgctggagttgcaaatcaatgcatactgggccacgcgccccagccatttcgtgttattgaacgcgcaaaaacttcgtgatacccagcacatgatgcagcaaatactgctgagccttgttcatgcgctgtacgaaggtaatccgcagccggtttttgccaatacggaaaaattgaacgatgctgtggaagagctgcgtcagttgctcaataaccaccatgacctgaaggttgtggaaacaccaatctatggttatgtgtggctgaacatggaaacggcgcatcagcttgagttgctatcgaatctgatttgccgggccttgcgcaaataa

SEQ ID NO: 6 - DNA, 621 bp, Artificial Sequence
  amino acid exporter gene rhtC
atgttgatgttatttctcaccgtcgccatggtgcacattgtggcgcttatgagccccggtcccgatttcttttttgtctctcagaccgctgtcagtcgttcccgtaaagaagcgatgatgggcgtgctgggcattacctgcggcgtaatggtttgggctgggattgcgctgcttggcctgcatttgattatcgaaaaaatggcctggctgcatacgctgattatggtgggcggtggcctgtatctctgctggatgggttaccagatgctacgtggtgcactgaaaaaagaggcggtttctgcacctgcgccacaggtcgagctggcgaaaagtgggcgcagtttcctgaaaggtttactgaccaatctcgctaatccgaaagcgattatctactttggctcggtgttctcattgtttgtcggtgataacgttggcactaccgcgcgctggggcatttttgcgctgatcattgtcgaaacgctggcgtggtttaccgtcgttgccagcctgtttgccctgccgcaaatgcgccgtggttatcaacgtctggcgaagtggattgatggttttgccggggcgttatttgccggatttggcattcatttgattatttcgcggtga

SEQ ID NO: 7 - DNA, 1185 bp, Artificial Sequence
  amino acid exporter gene emrD
atgaaaaggcaaagaaacgtcaatttgttattgatgttggtattactcgtggccgtcggtcagatggcgcaaaccatttatattccagctattgccgatatggcgcgcgatctcaacgtccgtgaaggggcggtgcagagcgtaatgggcgcttatctgctgacttacggtgtctcacagctgttttatggcccgatttccgaccgcgtgggccgccgaccggtgatcctcgtcggaatgtccatttttatgctggcaacgctggtcgcggtcacgacctccagtttgacggtgttgattgccgccagcgcgatgcaggggatgggcaccggcgttggcggcgtaatggcgcgtactttaccgcgagatttatatgaacggacacagttgcgccatgctaacagcctgttaaacatggggattctcgtcagtccgttgctcgcaccgctaatcggcggtctgctggatacgatgtggaactggcgcgcctgttatctctttttgttggttctttgtgctggtgtgaccttcagtatggcccgctggatgccggaaacgcgtccggtcgatgcaccgcgcacgcgcctgcttaccagttataaaacgcttttcggtaacagcggttttaactgttatttgctgatgctgattggcggtctggccgggattgccgcctttgaagcctgctccggcgtgctgatgggcgcggtgttagggctgagcagtatgacggtcagtattttgtttattctgccgattccggcagcgttttttggcgcatggtttgccggacgtcccaataaacgcttctccacgttaatgtggcagtcggttatctgctgcctgctggctggcttgctgatgtggatccccgactggtttggcgtgatgaatgtctggacgctgctcgttcccgccgcgctgttctttttcggtgccgggatgctgtttccgctggcgaccagcggcgcgatggagccgttccccttcctggcgggcacggctggcgcgctggtcggcggtctgcaaaacattggttccggcgtgctggcgtcgctctctgcgatgttaccgcaaaccggtcagggcagcctggggttgttgatgaccttaatgggattgttgatcgtgctgtgctggctgccgctggcgacgcggatgtcgcatcaggggcagcccgtttaa

SEQ ID NO: 8 - protein, 307 aa, Artificial Sequence
  amino acid exporter gene protein yicL
MGSTRKGMLNVLIAAVLWGSSGVCAQYIMEQSQMSSQFLTMTRLIFAGLILLTLSFVHGDKIFSIINNHKDAISLLIFSVVGALTVQLTFLLTIEKSNAATATVLQFLSPTIIVAWFSLVRKSRPGILVFCAILTSLVGTFLLVTHGNPTSLSISPAALFWGIASAFAAAFYTTYPSTLIARYGTLPVVGWSMLIGGLILLPFYARQGTNFVVNGSLILAFFYLVVIGTSLTFSLYLKGAQLIGGPKASILSCAEPLSSALLSLLLLGITFTLPDWLGTLLILSSVILISMDSRRRARKINRPARHK

SEQ ID NO: 9 - protein, 421 aa, Artificial Sequence
  amino acid exporter gene protein ydiN
MSQNKAFSTPFILAVLCIYFSYFLHGISVITLAQNMSSLAEKFSTDNAGIAYLISGIGLGRLISILFFGVISDKFGRRAVILMAVIMYLLFFFGIPACPNLTLAYGLAVCVGIANSALDTGGYPALMECFPKASGSAVILVKAMVSFGQMFYPMLVSYMLLNNIWYGYGLIIPGILFVLITLMLLKSKFPSQLVDASVTNELPQMNSKPLVWLEGVSSVLFGVAAFSTFYVIVVWMPKYAMAFAGMSEAEALKTISYYSMGSLVCVFIFAALLKKMVRPIWANVFNSALATITAAIIYLYPSPLVCNAGAFVIGFSAAGGILQLGVSVMSEFFPKSKAKVTSIYMMMGGLANFVIPLITGYLSNIGLQYIIVLDFTFALLALITAIIVFIRYYRVFIIPENDVRFGERKFCTRLNTIKHRG

SEQ ID NO: 10 - protein, 670 aa, Artificial Sequence
  amino acid exporter gene protein ydhK
MNASSWSLRNLPWFRATLAQWRYALRNTIAMCLALTVAYYLNLDEPYWAMTSAAVVSFPTVGGVISKSLGRIAGSLLGAIAALLLAGHTLNEPWFFLLSMSAWLGFCTWACAHFTNNVAYAFQLAGYTAAIIAFPMVNITEASQLWDIAQARVCEVIVGILCGGMMMMILPSSSDATALLTALKNMHARLLEHASLLWQPETTDAIRAAHEGVIGQILTMNLLRIQAFWSHYRFRQQNARLNALLHQQLRMTSVISSLRRMLLNWPSPPGATREILEQLLTALASSQTDVYTVARIIAPLRPTNVADYRHVAFWQRLRYFCRLYLQSSQELHRLQSGVDDHTRLPRTSGLARHTDNAEAMWSGLRTFCTLMMIGAWSIASQWDAGANALTLAAISCVLYSAVAAPFKSLSLLMRTLVLLSLFSFVVKFGLMVQISDLWQFLLFLFPLLATMQLLKLQMPKFAALWGQLIVFMGSFIAVTNPPVYDFADFLNDNLAKIVGVALAWLAFAILRPGSDARKSRRHIRALRRDFVDQLSRHPTLSESEFESLTYHHVSQLSNSQDALARRWLLRWGVVLLNCSHVVWQLRDWESRSDPLSRVRDNCISLLRGVMSERGVQQKSLAATLEELQRICDSLARHHQPAARELAAIIWRLYCSLSQLEQAPPQGTLAS

SEQ ID NO: 11 - protein, 655 aa, Artificial Sequence
  amino acid exporter gene protein aaeB
MGIFSIANQHIRFAVKLATAIVLALFVGFHFQLETPRWAVLTAAIVAAGTAFAAGGEPYSGAIRYRGFLRIIGTFIGCIAGLVIIIAMIRAPLLMILVCCIWAGFCTWISSLVRIENSYAWGLAGYTALIIVITIQPEPLLTPQFAVERCSEIVIGIVCAIMADLLFSPRSIKQEVDRELESLLVAQYQLMQLCIKHGDGEVVDKAWGDLVRRTTALQGMRSNLNMESSRWARANRRLKAINTLSLTLITQSCETYLIQNTRPELITDTFREFFDTPVETAQDVHKQLKRLRRVIAWTGERETPVTIYSWVAAATRYQLLKRGVISNTKINATEEEILQGEPEVKVESAERHHAMVNFWRTTLSCILGTLFWLWTGWTSGSGAMVMIAVVTSLAMRLPNPRMVAIDFIYGTLAALPLGLLYFLVIIPNTQQSMLLLCISLAVLGFFLGIEVQKRRLGSMGALASTINIIVLDNPMTFHFSQFLDSALGQIVGCVLAFTVILLVRDKSRDRTGRVLLNQFVSAAVSAMTTNVARRKENHLPALYQQLFLLMNKFPGDLPKFRLALTMIIAHQRLRDAPIPVNEDLSAFHRQMRRTADHVISARSDDKRRRYFGQLLEELEIYQEKLRIWQAPPQVTEPVNRLAGMLHKYQHALTDS

SEQ ID NO: 12 - protein, 352 aa, Artificial Sequence
  amino acid exporter gene protein yeeA
VRADKSLSPFEIRVYRHYRIVHGTRVALAFLLTFLIIRLFTIPESTWPLVTMVVIMGPISFWGNVVPRAFERIGGTVLGSILGLIALQLELISLPLMLVWCAAAMFLCGWLALGKKPYQGLLIGVTLAIVVGSPTGEIDTALWRSGDVILGSLLAMLFTGIWPQRAFIHWRIQLAKSLTEYNRVYQSAFSPNLLERPRLESHLQKLLTDAVKMRGLIAPASKETRIPKSIYEGIQTINRNLVCMLELQINAYWATRPSHFVLLNAQKLRDTQHMMQQILLSLVHALYEGNPQPVFANTEKLNDAVEELRQLLNNHHDLKVVETPIYGYVWLNMETAHQLELLSNLICRALRK

SEQ ID NO: 13 - protein, 206 aa, Artificial Sequence
  amino acid exporter gene protein rhtC
MLMLFLTVAMVHIVALMSPGPDFFFVSQTAVSRSRKEAMMGVLGITCGVMVWAGIALLGLHLIIEKMAWLHTLIMVGGGLYLCWMGYQMLRGALKKEAVSAPAPQVELAKSGRSFLKGLLTNLANPKAIIYFGSVFSLFVGDNVGTTARWGIFALIIVETLAWFTVVASLFALPQMRRGYQRLAKWIDGFAGALFAGFGIHLIISR

SEQ ID NO: 14 - protein, 394 aa, Artificial Sequence
  amino acid exporter gene protein emrD
MKRQRNVNLLLMLVLLVAVGQMAQTIYIPAIADMARDLNVREGAVQSVMGAYLLTYGVSQLFYGPISDRVGRRPVILVGMSIFMLATLVAVTTSSLTVLIAASAMQGMGTGVGGVMARTLPRDLYERTQLRHANSLLNMGILVSPLLAPLIGGLLDTMWNWRACYLFLLVLCAGVTFSMARWMPETRPVDAPRTRLLTSYKTLFGNSGFNCYLLMLIGGLAGIAAFEACSGVLMGAVLGLSSMTVSILFILPIPAAFFGAWFAGRPNKRFSTLMWQSVICCLLAGLLMWIPDWFGVMNVWTLLVPAALFFFGAGMLFPLATSGAMEPFPFLAGTAGALVGGLQNIGSGVLASLSAMLPQTGQGSLGLLMTLMGLLIVLCWLPLATRMSHQGQPV

SEQ ID NO: 15 - DNA, 25 bp, Artificial Sequence
  yddG H1F primer
caatgccgctactgttgttccagcc

SEQ ID NO: 16 - DNA, 25 bp, Artificial Sequence
  yddG H1R primer
cagtggtgcgtttttctaccgctat

SEQ ID NO: 17 - DNA, 22 bp, Artificial Sequence
  yddG H2F primer
aataactgccgggtctacggcc

SEQ ID NO: 18 - DNA, 33 bp, Artificial Sequence
  yddG H2R primer
aacgtattttctaaacgaattttaaacggcgtc

SEQ ID NO: 19 - DNA, 26 bp, Artificial Sequence
  yddG CF primer
cggaacagtatgtgcaggtgttacgg

SEQ ID NO: 20 - DNA, 26 bp, Artificial Sequence
  yddG CR primer
aacaaaccagttacaaccaccgcaac

SEQ ID NO: 21 - DNA, 28 bp, Artificial Sequence
  yicL_F primer
gcgagctcatgggttccaccagaaaggg

SEQ ID NO: 22 - DNA, 27 bp, Artificial Sequence
  yicL_R primer
gctctagatcacttatgccgcgccgga

SEQ ID NO: 23 - DNA, 33 bp, Artificial Sequence
  ydiN_F primer
gcgagctcatgtctcaaaataaggctttcagca

SEQ ID NO: 24 - DNA, 28 bp, Artificial Sequence
  ydiN_R primer
gctctagaggccatcaacccaatcaatt

SEQ ID NO: 25 - DNA, 33 bp, Artificial Sequence
  ydhK_F primer
gcgagctcatgaacgcatcgtcatggtccttgc

SEQ ID NO: 26 - DNA, 27 bp, Artificial Sequence
  ydhK_R primer
gctctagatcacttatgccgcgccgga

SEQ ID NO: 27 - DNA, 29 bp, Artificial Sequence
  aaeB_F primer
gcgagctcatgggtattttctccattgct

SEQ ID NO: 28 - DNA, 29 bp, Artificial Sequence
  aaeB_R primer
gctctagattttgacttaactatcggtca

SEQ ID NO: 29 - DNA, 25 bp, Artificial Sequence
  yeeA_F primer
gcgagctcgtgcgtgccgataagtc

SEQ ID NO: 30 - DNA, 26 bp, Artificial Sequence
  yeeA_R primer
gctctagattatttgcgcaaggcccg

SEQ ID NO: 31 - DNA, 31 bp, Artificial Sequence
  rhtC_F primer
gcgagctcatgttgatgttatttctcaccgt

SEQ ID NO: 32 - DNA, 28 bp, Artificial Sequence
  rhtC_R primer
gctctagactggcatcaccgcgaaataa

SEQ ID NO: 33 - DNA, 27 bp, Artificial Sequence
  emrD_F primer
gcgagctcatgaaaaggcaaagaaacg

SEQ ID NO: 34 - DNA, 27 bp, Artificial Sequence
  emrD_R primer
gctctagacggtgacgtgcgcttaaac

SEQ ID NO: 35 - DNA, 24 bp, Artificial Sequence
  Primer_F primer
gcgccgacatcataacggttctgg

SEQ ID NO: 36 - DNA, 23 bp, Artificial Sequence
  Primer_R primer
cgcaacgttcaaatccgctcccg

SEQ ID NO: 37 - DNA, 2013 bp, Artificial Sequence
  amino acid exporter gene ydhK 1945G
atgaacgcatcgtcatggtccttgcgcaatttgccctggttcagggccacgctggcgcaatggcgttatgcgttacgcaataccattgccatgtgtctggcgctgacggttgcctattatttaaatctggatgaaccctattgggcgatgacctcggctgcagtggttagctttcccaccgttggcggtgttatcagcaaaagcctcggacgcatcgctggcagtttgctcggagccattgcggcactgcttcttgccgggcatacgctcaatgagccgtggttttttctattgagcatgtcggcgtggcttggcttttgtacctgggcctgtgcgcacttcacgaataacgtcgcgtatgcatttcaactggcgggctacacggctgccatcatcgcctttccgatggttaatattactgaggccagccagctgtgggatatcgctcaggcgcgcgtttgcgaggtaatagtcggtattttgtgcggcggcatgatgatgatgatcctgccgagcagttccgatgctactgcccttttaaccgcattgaaaaacatgcacgcccgattactggaacatgccagtttactctggcagcctgaaacaaccgatgccattcgtgcagcacatgaaggggtgattgggcagatactgaccatgaatttgctgcgtatccaggctttctggagccactatcgttttcgccagcaaaacgcgcgccttaatgcgctgctccaccagcaattacgtatgaccagtgtcatctccagcctgcgacgtatgttgctcaactggccctcaccgccaggtgccacacgagaaattctcgaacagttgctgacggcgctcgccagttcgcaaacagatgtttacaccgtcgcacgtattatcgccccgctacgcccgaccaacgtcgccgactatcggcacgtcgccttctggcagcgactacgttatttttgccgcctttatctgcaaagtagtcaggaattacatcgtctgcaaagcggtgtagatgatcataccagactcccacggacatccggcctggctcgtcataccgataacgccgaagctatgtggagcgggctgcgtacattttgtacgttgatgatgattggcgcatggagtattgcttcgcaatgggatgccggtgccaatgcattaacgctggcagcaattagctgcgtactctactccgccgtcgcagcaccgtttaagtcgttgtcacttctgatgcgcacgctggtgttactttcgctattcagctttgtggtcaaatttggtctgatggtccagattagcgatctgtggcaatttttactgtttctctttccactgctggcgacaatgcagcttcttaaattgcagatgccaaaatttgccgcattgtgggggcaactgattgtttttatgggttcttttatcgctgtcactaatcccccggtgtatgattttgctgattttcttaacgataatctggcaaaaatcgttggcgtcgcgttggcgtggttagcgttcgccattctgcgtccaggatcggatgctcgtaaaagccgccgccatattcgcgcgctgcgccgggattttgtcgatcagctaagccgccatccaacactgagtgaaagcgaatttgaatcgctcacttatcatcacgtcagtcagttgagtaacagccaggatgcgctggctcgccgttggttattacgctggggtgtagtgctgctgaactgttctcatgttgtctggcaattgcgcgactgggaatcgcgttccgatccgttatcgcgagtacgggataactgtatttcactgttgcggggagtgatgagtgagcgtggcgttcagcaaaaatcactggcggccacacttgaagaattacagcggatttgcgacagccttgcccgtcatcatcaacctgccgcccgtgagctggcggcaattgtctggcggctgtactgctcgctttcgcaacttgagcaagcaccaccgcaaggtacgctggcctcttaa

SEQ ID NO: 38 - protein, 670 aa, Artificial Sequence
  amino acid exporter gene protein ydhK 649V
MNASSWSLRNLPWFRATLAQWRYALRNTIAMCLALTVAYYLNLDEPYWAMTSAAVVSFPTVGGVISKSLGRIAGSLLGAIAALLLAGHTLNEPWFFLLSMSAWLGFCTWACAHFTNNVAYAFQLAGYTAAIIAFPMVNITEASQLWDIAQARVCEVIVGILCGGMMMMILPSSSDATALLTALKNMHARLLEHASLLWQPETTDAIRAAHEGVIGQILTMNLLRIQAFWSHYRFRQQNARLNALLHQQLRMTSVISSLRRMLLNWPSPPGATREILEQLLTALASSQTDVYTVARIIAPLRPTNVADYRHVAFWQRLRYFCRLYLQSSQELHRLQSGVDDHTRLPRTSGLARHTDNAEAMWSGLRTFCTLMMIGAWSIASQWDAGANALTLAAISCVLYSAVAAPFKSLSLLMRTLVLLSLFSFVVKFGLMVQISDLWQFLLFLFPLLATMQLLKLQMPKFAALWGQLIVFMGSFIAVTNPPVYDFADFLNDNLAKIVGVALAWLAFAILRPGSDARKSRRHIRALRRDFVDQLSRHPTLSESEFESLTYHHVSQLSNSQDALARRWLLRWGVVLLNCSHVVWQLRDWESRSDPLSRVRDNCISLLRGVMSERGVQQKSLAATLEELQRICDSLARHHQPAARELAAIVWRLYCSLSQLEQAPPQGTLAS

SEQ ID NO: 39 - DNA, 1185 bp, Artificial Sequence
  amino acid exporter gene emrD 1071G
atgaaaaggcaaagaaacgtcaatttgttattgatgttggtattactcgtggccgtcggtcagatggcgcaaaccatttatattccagctattgccgatatggcgcgcgatctcaacgtccgtgaaggggcggtgcagagcgtaatgggcgcttatctgctgacttacggtgtctcacagctgttttatggcccgatttccgaccgcgtgggccgccgaccggtgatcctcgtcggaatgtccatttttatgctggcaacgctggtcgcggtcacgacctccagtttgacggtgttgattgccgccagcgcgatgcaggggatgggcaccggcgttggcggcgtaatggcgcgtactttaccgcgagatttatatgaacggacacagttgcgccatgctaacagcctgttaaacatggggattctcgtcagtccgttgctcgcaccgctaatcggcggtctgctggatacgatgtggaactggcgcgcctgttatctctttttgttggttctttgtgctggtgtgaccttcagtatggcccgctggatgccggaaacgcgtccggtcgatgcaccgcgcacgcgcctgcttaccagttataaaacgcttttcggtaacagcggttttaactgttatttgctgatgctgattggcggtctggccgggattgccgcctttgaagcctgctccggcgtgctgatgggcgcggtgttagggctgagcagtatgacggtcagtattttgtttattctgccgattccggcagcgttttttggcgcatggtttgccggacgtcccaataaacgcttctccacgttaatgtggcagtcggttatctgctgcctgctggctggcttgctgatgtggatccccgactggtttggcgtgatgaatgtctggacgctgctcgttcccgccgcgctgttctttttcggtgccgggatgctgtttccgctggcgaccagcggcgcgatggagccgttccccttcctggcgggcacggctggcgcgctggtcggcggtctgcaaaacattggttccggcgtgctggcgtcgctctctgcgatgttgccgcaaaccggtcagggcagcctggggttgttgatgaccttaatgggattgttgatcgtgctgtgctggctgccgctggcgacgcggatgtcgcatcaggggcagcccgtttaa